(19) United States
(12) Patent Application Publication
(10) Pub. No.: US 2026/0083339 A1
(43) Pub. Date: Mar. 26, 2026

(54) DETECTION OF RIGHT VENTRICULAR SYSTOLIC DYSFUNCTION

(71) Applicant: Becton, Dickinson and Company, Franklin Lakes, NJ (US)

(72) Inventors: Kevin James Moses, Tustin, CA (US); Cristhian M. Potes Blandon, Rancho Santa Margarita, CA (US); Christine Lee, Long Beach, CA (US)

(21) Appl. No.: 19/400,900

(22) Filed: Nov. 25, 2025

Related U.S. Application Data
(63) Continuation of application No. PCT/US2024/031297, filed on May 28, 2024.
(60) Provisional application No. 63/505,679, filed on Jun. 1, 2023.

(51) Int. Cl. A61B 5/0215; A61B 5/00

(52) U.S. Cl. CPC A61B 5/0215; A61B 5/6852; A61B 5/742; A61B 5/746

(57) ABSTRACT

A system for monitoring right ventricular systolic dysfunction includes a hemodynamic sensor that produces a hemodynamic sensor signal representative of a right ventricular pressure waveform of the patient, a display, one or more processors, and computer-readable memory. The computer-readable memory is encoded with instructions that, when executed by the one or more processors, cause the system to receive the hemodynamic sensor signal representative of the right ventricular pressure waveform of the patient, extract right ventricular peak systolic pressure and right ventricular end diastolic pressure from the right ventricular pressure waveform of the patient, determine a ratio of right ventricular peak systolic pressure over right ventricular end diastolic pressure, and output the ratio of right ventricular peak systolic pressure over right ventricular end diastolic pressure to the display for monitoring the presence of right ventricular systolic dysfunction based on the ratio of peak systolic pressure over end diastolic pressure.

### CROSS-REFERENCE TO RELATED APPLICATION(S)

This application is a continuation of International Application No. PCT/US2024/031297, filed May 28, 2024, which claims the benefit of U.S. Provisional Application No. 63/505,679, filed Jun. 1, 2023, and entitled “DETECTION OF RIGHT VENTRICULAR SYSTOLIC DYSFUNCTION,” the disclosure of which is hereby incorporated by reference in its entirety.

### BACKGROUND

The present disclosure relates generally to right ventricular (RV) systolic dysfunction, and in particular, to detection of RV systolic dysfunction in a patient.

RV systolic dysfunction is a major issue in the ICU and OR, especially in patients coming off of cardiopulmonary bypass. RV systolic dysfunction can indicate future cardiovascular events, such as heart attack and heart failure. Current methods to detect RV systolic dysfunction utilize ultrasound technology, which requires a trained physician and the availability of resources. Means of detecting RV systolic dysfunction that requires less resources would allow for improved patient care.

### SUMMARY

A system for monitoring right ventricular systolic dysfunction in a patient includes a hemodynamic sensor that produces, on an ongoing basis, a hemodynamic sensor signal representative of a right ventricular pressure waveform of the patient, a display, one or more processors, and computer-readable memory. The computer-readable memory is encoded with instructions that, when executed by the one or more processors, cause the system to receive the hemodynamic sensor signal representative of the right ventricular pressure waveform of the patient, extract right ventricular peak systolic pressure and right ventricular end diastolic pressure from the right ventricular pressure waveform of the patient, determine a ratio of right ventricular peak systolic pressure over right ventricular end diastolic pressure, and output the ratio of right ventricular peak systolic pressure over right ventricular end diastolic pressure to the display for monitoring the presence of right ventricular systolic dysfunction based on the ratio of peak systolic pressure over end diastolic pressure.

A method for detecting right ventricular systolic dysfunction in a patient includes receiving, by a hemodynamic monitor, sensed hemodynamic data representative of a right ventricular pressure waveform of the patient; performing, by the hemodynamic monitor, waveform analysis of the hemodynamic data to determine right ventricular peak systolic pressure and right ventricular end diastolic pressure from the right ventricular pressure waveform of the patient; and determining, by the hemodynamic monitor, a ratio of right ventricular peak systolic pressure over right ventricular end diastolic pressure to detect right ventricular systolic dysfunction.

A system for detecting right ventricular systolic dysfunction in a patient includes a hemodynamic sensor that produces a hemodynamic sensor signal representative of a right ventricular pressure waveform of the patient, a display, one or more processors, and computer-readable memory. The computer-readable memory is encoded with instructions that, when executed by the one or more processors, cause the system to receive the hemodynamic sensor signal representative of the right ventricular pressure waveform of the patient, extract right ventricular peak systolic pressure and right ventricular end diastolic pressure from the right ventricular pressure waveform of the patient;, determine a ratio of right ventricular peak systolic pressure over right ventricular end diastolic pressure, and output the ratio of right ventricular peak systolic pressure over right ventricular end diastolic pressure to the display for detecting right ventricular systolic dysfunction based on the ratio of peak systolic pressure over end diastolic pressure.

### BRIEF DESCRIPTION OF THE DRAWINGS

FIG. 1 is a perspective view of an example hemodynamic monitor that analyzes an RV pressure waveform and provides a ratio of RV peak systolic pressure over RV end diastolic pressure that detects RV systolic dysfunction in a patient.

FIG. 2 is a perspective view of a catheter that can be inserted in a patient and connected to one or more hemodynamic sensors.

FIG. 3 is a perspective view of an example minimally invasive pressure sensor for sensing hemodynamic data representative of pulmonary artery pressure or right ventricular pressure of a patient.

FIG. 4 is a perspective view of an oximetry module for receiving oximetry data from a catheter inserted within a patient.

FIG. 5A is a schematic view of a tissue oximetry sensor to determine oxygen saturation within cerebral tissue of a patient.

FIG. 5B is a tissue oximetry module that can be used in conjunction with a tissue oximetry sensor to determine oxygen saturation within cerebral tissue of a patient.

FIG. 6 is a block diagram illustrating an example hemodynamic monitoring system that analyzes an RV pressure waveform and provides a ratio of RV peak systolic pressure over RV end diastolic pressure to detect RV systolic dysfunction for a patient based on the hemodynamic data.

FIG. 7 is a graph illustrating an example trace of an RV pressure waveform including example indicia corresponding to the RV peak systolic pressure and the RV end diastolic pressure.

FIG. 8 is a flow diagram illustrating example operations to extract a set of features from an RV pressure waveform of a patient for detecting and assessing the progression of RV systolic dysfunction.

### DETAILED DESCRIPTION

In general, the present disclosure describes a hemodynamic monitoring system that utilizes a Swan Ganz catheter and a hemodynamic sensor to generate a right ventricular (RV) pressure waveform for continuously monitoring a ratio of RV peak systolic pressure over RV end diastolic pressure to detect and assess the progression of RV systolic dysfunction in a patient in e.g., an operating room (OR), an intensive care unit (ICU), or other patient care environment. The system is easier and more cost-effect to execute and can inform medical personnel of the presence and severity of RV systolic dysfunction in the patient to assist with patient care.

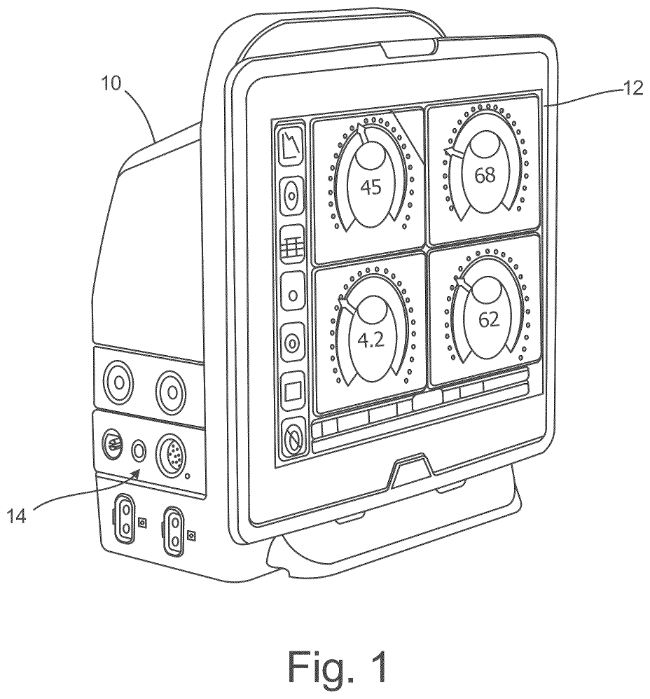

FIG. 1 is a perspective view of hemodynamic monitor 10 that analyzes an RV pressure waveform and provides a ratio of RV peak systolic pressure over RV end diastolic pressure to detect RV systolic dysfunction in a patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores RV systolic dysfunction software code, which is executable to determine RV systolic dysfunction in a patient based on sensed hemodynamic data of the patient. Hemodynamic monitor 10 can receive sensed hemodynamic data representative of a ventricular pressure waveform of the patient, such as via one or more hemodynamic sensors connected to hemodynamic monitor 10 via I/O connectors 14. Hemodynamic monitor 10 executes the RV systolic dysfunction software code to obtain, using the sensed hemodynamic data and RV systolic dysfunction profiling parameters (e.g., input features), the severity of RV systolic dysfunction for a patient, as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

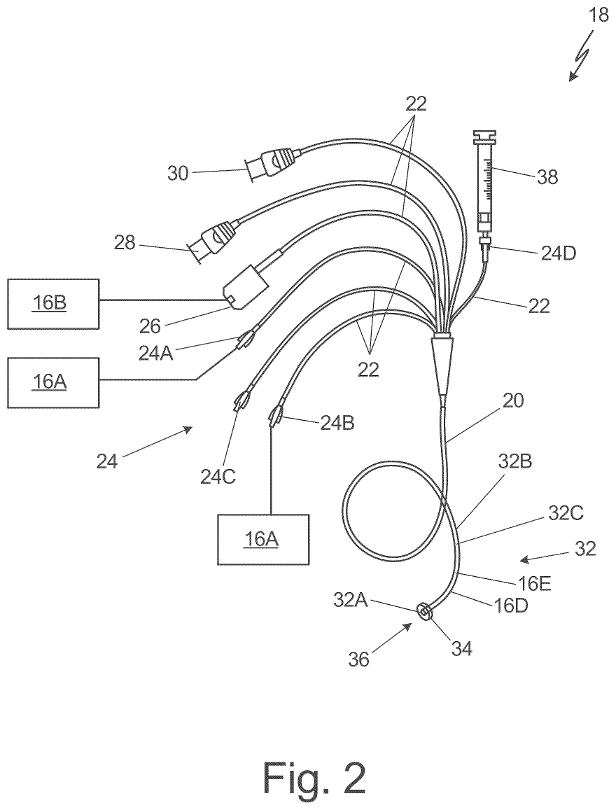

Hemodynamic monitor 10 receives hemodynamic data from a patient via one or more hemodynamic sensors 16A, 16B, 16C, and 16D (collectively hemodynamic sensors 16) (shown in FIGS. 2-5B). In response to receiving hemodynamic data of the patient, hemodynamic monitor 10 executes the RV systolic dysfunction software code to determine RV systolic dysfunction in a patient and display the presence, or lack thereof, and severity of RV systolic dysfunction on display 12. In some examples, hemodynamic monitor 10 can invoke a sensory alarm, such as an audible alarm, a haptic alarm, or other sensory alarm in response to determining that RV systolic dysfunction is occurring in the patient. Accordingly, hemodynamic monitor 10 can provide a warning to medical personnel of RV systolic dysfunction in the patient as well as an increase or decrease in RV systolic dysfunction.

FIG. 2 depicts catheter 18 that can be connected to one or more hemodynamic sensors 16 for providing hemodynamic data to hemodynamic monitor 10. For example, catheter 18 may be connected to one or more pressure-sensing hemodynamic sensors 16A for detecting right ventricular pressure, pulmonary artery pressure, or both right ventricular and pulmonary artery pressures of the patient. Additionally, catheter 18 may interface with oximetry module 16B for sensing mixed venous oxygen saturation of the patient. Protected by sheath 20, catheter 18 includes multiple lumens 22 that place fluid connectors 24, optical connector 26, thermistor connector 28, and thermal filament connector 30 in communication with one of ports 32, an embedded hemodynamic sensor 16D (e.g., a thermistor), or an embedded hemodynamic sensor 16E (e.g., a thermal filament). To facilitate insertion of catheter 18 within patient, or for certain hemodynamic measurements, catheter 18 includes balloon 34 located at tip 36 of catheter 18.

As shown in FIG. 2, catheter 18 includes distal port connector 24A communicating with port 32A at tip 36. Proximal injectate connector 24B communicates with proximal port 32B disposed approximately 30 cm from tip 36 and can be used for dispensing fluids and drugs into the patient's heart. Right ventricular pacing connector 24C communicates with right ventricle port 32C, which may be spaced approximately 19 cm from tip 36 or approximately 12 to 13 cm from tip 36. Connector 24C can be used for sensing a right ventricular pressure of the patient's heart. Thermistor connector 28 electrically connects to hemodynamic sensor 16D (e.g., the thermistor) installed near tip 36 of catheter 18 for measuring core blood temperature within the pulmonary artery. In some embodiments of catheter 18, thermal filament connector 30 electrically connects to hemodynamic sensor (16E) (e.g., the thermal filament) embedded within catheter 18 located within the patient's right ventricle. In some examples, catheter 18 does not include a thermal filament or corresponding thermal filament connector 30. Balloon connector 24D communicates with balloon 34 and with the use of syringe 38 can be used to inflate and deflate balloon 34.

After insertion into the patient, e.g., via an introducer, distal port connector 24A and right ventricular pacing connector 24C can be connected to separate pressure transducer sensors 16A. A first pressure transducer sensor 16A provides pulmonary artery pressure waveform data to hemodynamic monitor 10 sensed at distal port 32A located within the pulmonary artery while a second pressure transducer sensor 16A provides right ventricular pressure waveform data sensed at right ventricle port 32C located within the right ventricle of the patient's heart. Blood oxygen saturation data within the pulmonary artery can be provided by oximetry module 16B based on light pulses emitted from oximetry module 16B into the pulmonary artery and reflected light returns received by oximetry module 16B via optical connector 26 of catheter 18. Additionally, utilizing thermal filament connector 30 and thermistor connector 28 and associated cabling, hemodynamic monitor 10 can receive cardiac output data of the patient using, for example, a thermal dilution technique. The cardiac output measured via the thermal filament and corresponding thermal filament connector 30 can be considered a continuous cardiac output. If catheter 18 does not include a thermal filament, cardiac output can be determined using thermistor connector 28 after injecting a fluid bolus (or a set of boluses) of known volume and temperature via proximal injectate port 32B using the thermal dilution technique. The cardiac output measured via the thermistor and corresponding thermistor connector 28 after injection of the fluid bolus can be considered an intermittent cardiac output. Intermittent cardiac output measurements can be obtained at a frequency that is on the order of, e.g., minutes, hours, several hours, or even longer intervals, depending on the level of monitoring a patient requires. For example, a clinician may administer a bolus set of 3-4 fluid boluses, where one fluid bolus of the set is administered approximately every minute such that the complete bolus set lasts around three minutes. In one example, fluid boluses can be administered very frequently, such as every minute or every few minutes, when a clinician is assessing a patient's responsiveness to medication or another medical intervention. In another example, fluid boluses can be administered less frequently, such as every hour, every six hours, etc., if a patient is relatively stable in the ICU. Catheter 18 is one example of a catheter that can be used to measure right ventricular pressure waveform data. In other examples, any catheter configured to measure right ventricular pressure waveform data can be used.

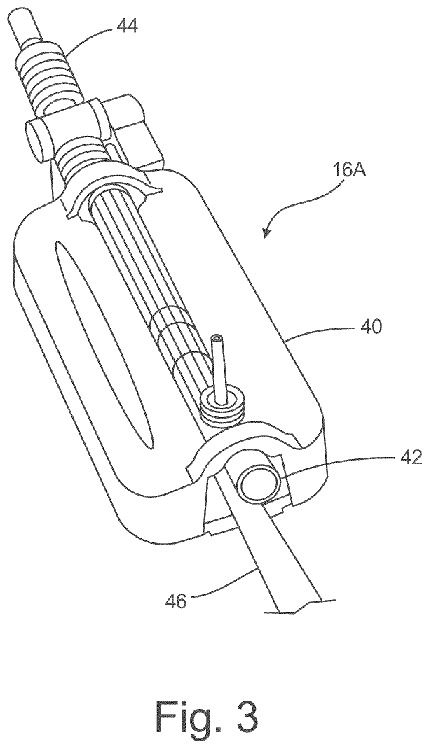

FIG. 3 is a perspective view of hemodynamic sensor 16A that can be attached to a patient for sensing hemodynamic data representative of right ventricular pressure or pulmonary artery pressure of the patient. As illustrated in FIG. 3, hemodynamic sensor 16A includes housing 40, fluid input port 42, catheter-side fluid port 44, and I/O cable 46. Fluid input port 42 is configured to be connected via tubing or other hydraulic connection to a fluid source, such as a saline bag or other fluid input source. Catheter-side fluid port 44 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 46 is configured to connect to hemodynamic monitor 10 via, e.g., one or more of I/O connectors 14 (FIG. 1). Housing 40 of hemodynamic sensor 16A encloses one or more pressure transducers, communication circuitry, processing circuity, and corresponding electronic components to sense fluid pressure corresponding to right ventricular pressure or pulmonary artery pressure of the patient that is transmitted to hemodynamic monitor 10 (FIG. 1) via I/O cable 46.

In operation, a column of fluid (e.g., saline solution) is introduced from a fluid source (e.g., a saline bag) through hemodynamic sensor 16A via fluid input port 42 to catheter-side fluid port 44 toward the catheter inserted into the patient. Right ventricular pressure or pulmonary artery pressure is communicated through the fluid column to pressure sensors located within housing 40 which sense the pressure of the fluid column. Hemodynamic sensor 16A translates the sensed pressure of the fluid column to an electrical signal via the pressure transducers and outputs the corresponding electrical signal to hemodynamic monitor 10 (FIG. 1) via I/O cable 46. Hemodynamic sensor 16 therefore transmits analog sensor data (or a digital representation of the analog sensor data) to hemodynamic monitor 10 (FIG. 1) that is representative of substantially continuous beat-to-beat monitoring of the right ventricular pressure or pulmonary artery pressure of the patient.

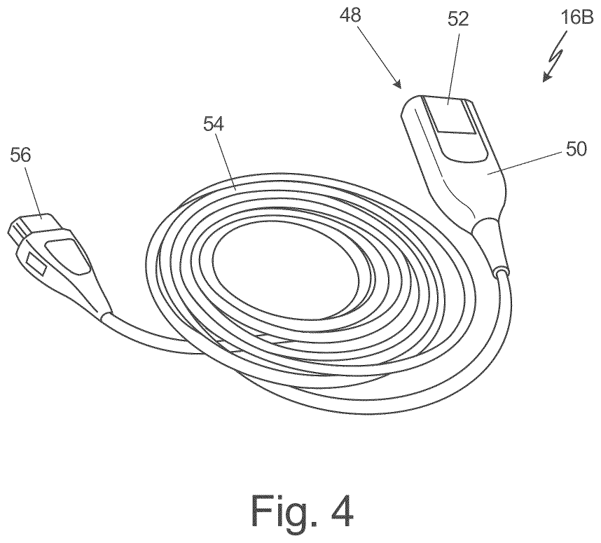

FIG. 4 depicts oximetry module 16B used for receiving oximetry data from a catheter inserted within a patient. As depicted in FIG. 4, hemodynamic sensor 16B includes an optical transmitter and an optical receiver arranged to communicate to a catheter via input/output connector 48 installed within housing 50 and accessible via protective door 52. Within housing 50, hemodynamic sensor 16B, as depicted by FIG. 4, includes communication circuitry, processing circuity, and corresponding electronic components to sense blood oxygen saturation data derived from optical light emissions transmitted via a catheter into a patient and corresponding light returns received from the patient via the catheter. An electrical signal indicative of the patient blood oxygen saturation levels is transmitted to hemodynamic monitor 10 via cable 54 and connector 56, which interfaces with one of I/O connectors 14 (FIG. 1).

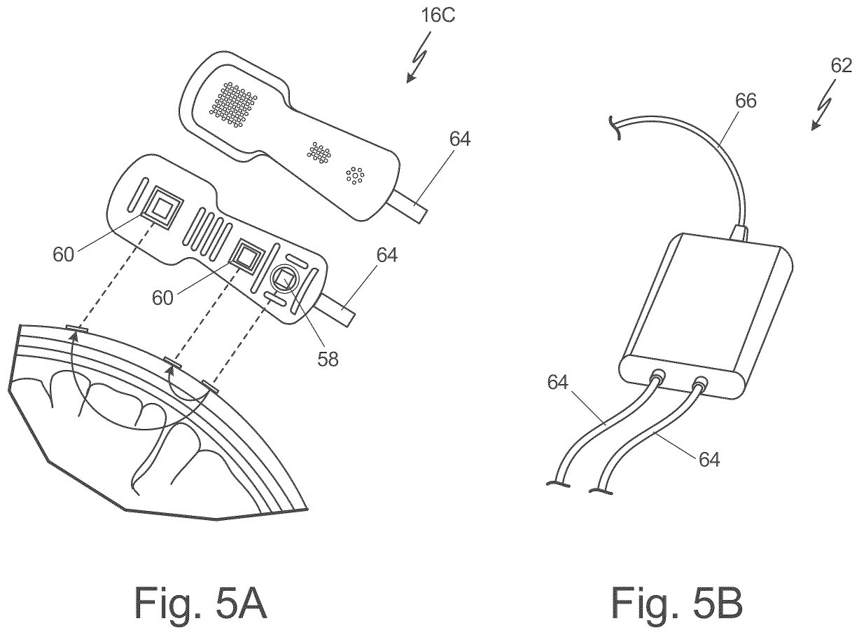

FIG. 5A is a schematic view of tissue oximetry sensor 16C for determining blood oxygen saturation within cerebral tissue of the patient. FIG. 5B is an isometric view of tissue oximetry module 62 that can be used in conjunction with tissue oximetry sensor 16C to determine oxygen saturation within cerebral tissue of the patient. The determined blood oxygen saturation within cerebral tissue can be provided to hemodynamic monitor 10. Tissue oximetry sensor 16C includes light emitter 58 and one or more detectors 60. Oximetry module 62 depicted in FIG. 5B connects to one or more tissue oximetry sensors 16C via cable 64 and includes communication circuitry, processing circuity, and corresponding electronic components to cause tissue oximetry sensor 16C or oximetry sensors 16C to emit light pulses into cerebral tissue of the patient. Light returns received by one or more detectors 60 of each tissue oximetry sensor 16C are received via cables 64 and processed by oximetry module 62. An electrical signal indicative of the patient tissue oxygen saturation levels is transmitted to hemodynamic monitor 10 via cable 66, which interfaces with one of I/O connectors 14 (FIG. 1).

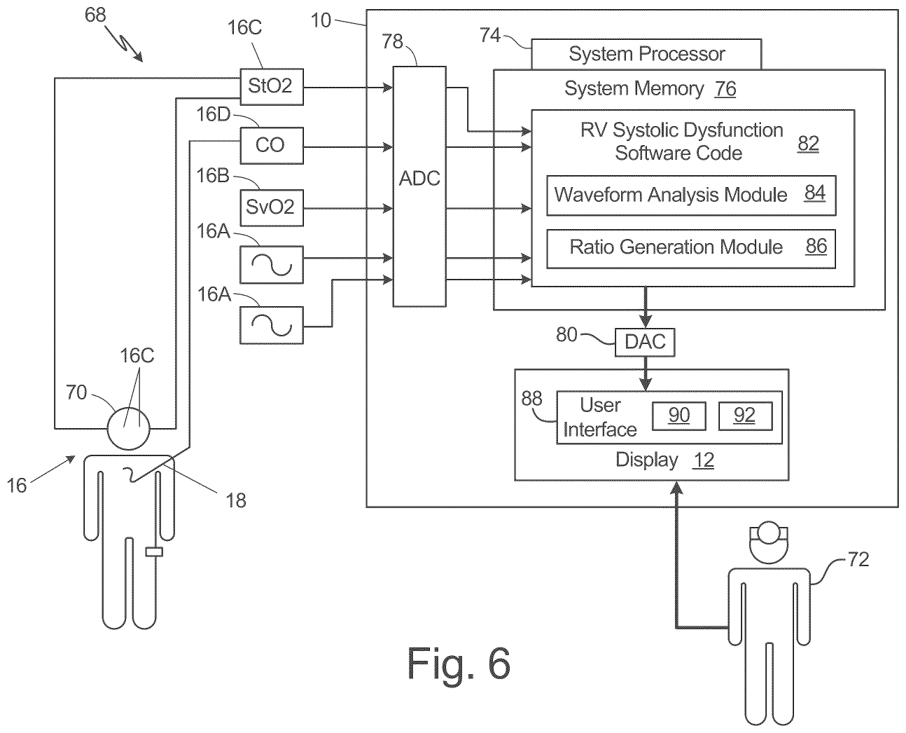

FIG. 6 is a block diagram of hemodynamic monitoring system 68 that analyses an RV pressure waveform to determine RV peak systolic pressure and RV end diastolic pressure and provide a ratio of RV peak systolic pressure over RV end diastolic pressure to detect RV systolic dysfunction for a patient based on the hemodynamic data. As illustrated in FIG. 6, hemodynamic monitoring system 68 includes hemodynamic monitor 10 and hemodynamic sensors 16 (including hemodynamic sensors 16A, 16B, 16C, 16D, and 16E). Hemodynamic monitoring system 68 can be implemented within a patient care environment, such as an ICU, an OR, or other patient care environment. As illustrated in FIG. 6, the patient care environment can include patient 70 and healthcare worker 72 trained to utilize hemodynamic monitoring system 68.

Hemodynamic monitor 10, as described above with respect to FIG. 1, can be an integrated hardware unit including system processor 74, system memory 76, display 12, analog-to-digital converter (ADC) 78, and digital-to-analog converter (DAC) 80. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. Likewise, at least a portion of data processing within hemodynamic monitoring system 68 can occur via a smart cable that is connected between a catheter or sensor and hemodynamic monitor 10. In general, though illustrated and described in the example of FIG. 6 as an integrated hardware unit, it should be understood that hemodynamic monitor 10 can include any combination of devices and components that are electrically, communicatively, or otherwise operatively connected to perform functionality attributed herein to hemodynamic monitor 10.

As illustrated in FIG. 6, system memory 76 stores RV systolic dysfunction software code 82. RV systolic dysfunction software code 82 includes waveform analysis module 84 and ratio generation module 86. Display 12 provides user interface 88, which includes control elements 90 that enable user interaction with hemodynamic monitor 10 and/or other components of hemodynamic monitoring system 68. User interface 88, as illustrated in FIG. 6, also provides sensory alarm 92 to provide warning to medical personnel of an increase in RV systolic dysfunction of patient 70.

Hemodynamic sensors 16 can be attached to patient 70 to sense hemodynamic data representative of a right ventricular pressure waveform, a pulmonary artery pressure waveform, blood oxygen saturation (SvO2), cerebral tissue oxygen saturation (StO2), or cardiac output (CO) of patient 70, or any combination of these hemodynamic data. Hemodynamic sensors 16 are operatively connected to hemodynamic monitor 10 (e.g., electrically and/or communicatively connected via wired or wireless connection, or both) to provide the sensed hemodynamic data to hemodynamic monitor 10. In some examples, hemodynamic sensors 16 provide the hemodynamic data of patient 70 to hemodynamic monitor 10 as an analog signal, which is converted by ADC 80 to digital hemodynamic data representative of the right ventricular pressure waveform. In other examples, hemodynamic sensors 16 can provide the sensed hemodynamic data to hemodynamic monitor 10 in digital form, in which case hemodynamic monitor 10 may not include or utilize ADC 78. In yet other examples, hemodynamic sensors 16 can provide the hemodynamic data of patient 70 to hemodynamic monitor 10 as an analog signal, which is analyzed in its analog form by hemodynamic monitor 10.

Hemodynamic sensors 16 can include one or more non-invasive, minimally invasive, or invasive sensor attached to patient 70. For instance, hemodynamic sensors 16 can take the form of invasive hemodynamic sensor 16A, such as second pressure transducer 16A that provides right ventricular pressure waveform data sensed at right ventricle port 32C located within the right ventricle of the heart of patient 70 (FIG. 3). Hemodynamic sensors 16 can take the form of invasive hemodynamic sensor 16B, such as oximetry module 16B that provides blood oxygen saturation data within the pulmonary artery based on light pulses emitted from module 16B into the pulmonary artery and reflected, returned, and received by module 16B via optical connector 26 of catheter 18 (FIG. 4). Further, hemodynamic sensors 16 can take the form of non-invasive hemodynamic sensor 16C, such as tissue oximetry sensor 16C that provides oxygen saturation data within cerebral tissue of patient 70 (FIGS. 5A and 5B). In some examples, hemodynamic sensors 16 can be attached non-invasively at an extremity of patient 70, such as a forehead, a wrist, an arm, a finger, an ankle, a toe, or other extremity of patient 70. Hemodynamic sensors 16 can also take the form of other invasive, minimally invasive, or non-invasive hemodynamic sensors.

In certain examples, hemodynamic sensors 16 can be configured to sense right ventricular pressure, pulmonary artery pressure, or both right ventricular and pulmonary artery pressures of patient 70. In some instances, hemodynamic sensors 16 may also be used to sense cardiac output of the patient, blood oxygen saturation within the pulmonary artery, or both cardiac output and blood oxygen saturation in addition to right ventricular and pulmonary artery pressure waveforms. For instance, one or more hemodynamic sensors 16 can be attached to patient 70 via a radial arterial catheter inserted into an arm of patient 70. In other examples, one or more of hemodynamic sensors 16 can be attached to patient 70 via a femoral arterial catheter inserted into a leg of patient 70. In other examples, one or more of hemodynamic sensors 16 may provide tissue oxygen saturation levels within cerebral tissue of patient 70 via an oximetry sensor attached to a forehead of patient 70. Such techniques can similarly enable multiple hemodynamic sensors 16 to provide substantially continuous beat-to-beat monitoring of the right ventricular pressure and pulmonary artery pressure as well as monitoring of cardiac output, blood oxygen saturation, and tissue oxygen saturation of patient 70, or any combination of these hemodynamic data, over an extended period of time, such as minutes or hours.

System processor 74 executes RV systolic dysfunction software code 82, which implements waveform analysis module 84 and ratio generation module 86 to extract and utilize features of the RV pressure waveform for detecting and assessing RV systolic dysfunction in patient 70. Examples of system processor 74 can include any one or more of a microprocessor, a controller, a digital signal processor (DSP), an application specific integrated circuit (ASIC), a field-programmable gate array (FPGA), or other equivalent discrete or integrated logic circuitry.

System memory 76 can be configured to store information within hemodynamic monitor 10 during operation. System memory 76, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 76 can include volatile and non-volatile computer-readable memories. Examples of volatile memories can include random access memories (RAM), dynamic random-access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 88 can include graphical and/or physical control elements that enable user input to interact with hemodynamic monitor 10 and/or other components of hemodynamic monitoring system 68. In some examples, user interface 88 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 88 can take the form of and/or include physical control elements, such as a physical buttons, keys, knobs, or other physical control elements configured to receive user input to interact with components of hemodynamic monitoring system 68.

In operation, hemodynamic sensor 16A is connected to hemodynamic monitor 10 and catheter 18 (FIG. 2). Hemodynamic sensor 16A senses hemodynamic data representative of a right ventricular pressure waveform of patient 70. Hemodynamic sensor 16A provides the hemodynamic data (e.g., as analog sensor data), to hemodynamic monitor 10. ADC 78 converts the analog hemodynamic data to digital hemodynamic data representative of the right ventricular pressure waveform of patient 70.

System processor 74 executes RV systolic dysfunction software code 82 to detect, using the received hemodynamic data, the presence and severity of RV systolic dysfunction for patient 70. For instance, system processor 74 can execute RV systolic dysfunction software code 82 to perform waveform analysis of the received hemodynamic data. RV systolic dysfunction software code 82 uses waveform analysis module 84 to determine features of the RV pressure waveform, including RV peak systolic pressure and RV end diastolic pressure. Subsequently, system processor 74 further executes RV systolic dysfunction software code 82 to generate a ratio of RV peak systolic pressure over RV end diastolic pressure via ratio generation module 86. The ratio of RV peak systolic pressure over RV end diastolic pressure is used to detect and assess RV systolic dysfunction in patient 70.

RV systolic dysfunction is identified via the ratio of RV peak systolic pressure over RV end diastolic pressure. An increase in end diastolic pressure and/or a decrease in systolic pressure occurs as a result of RV systolic dysfunction. As such, RV systolic dysfunction causes a decrease in the ratio of RV peak systolic pressure over RV end diastolic pressure. The lower the ratio of RV peak systolic pressure over RV end diastolic pressure, the more severe RV systolic dysfunction. A ratio of RV peak systolic pressure over RV end diastolic pressure greater than 3 signifies that no RV systolic dysfunction is occurring. A ratio of RV peak systolic pressure over RV end diastolic pressure of 2.5 or below signifies that RV systolic dysfunction is occurring. A ratio of RV peak systolic pressure over RV end diastolic pressure of 2 or below signifies that severe RV systolic dysfunction is occurring.

Hemodynamic monitor 10 uses hemodynamic sensors 16 to help assess the hemodynamic state of the patient. Accordingly, hemodynamic monitor 10 informs healthcare worker 72 of the presence of RV systolic dysfunction in patient 70, thereby enabling timely and effective patient care. Unlike ultrasound, which cannot continuously assess right ventricular performance, hemodynamic monitor 10 continuously generates data (about every 2 seconds, or with each heartbeat), allowing for continuous monitoring of RV systolic dysfunction. As such, hemodynamic monitor 10 enables real-time updates to medical personnel of RV systolic dysfunction. A trained physician is not required to utilize hemodynamic monitor 10 to detect RV systolic dysfunction, unlike ultrasound. As such, hemodynamic monitor 10 requires less resources and expertise to monitor RV systolic dysfunction than ultrasound methods, resulting in an easier and more cost-effective approach. Further, hemodynamic monitor 10 increases accuracy, as methods using ultrasound have a large variation between physicians and hospitals.

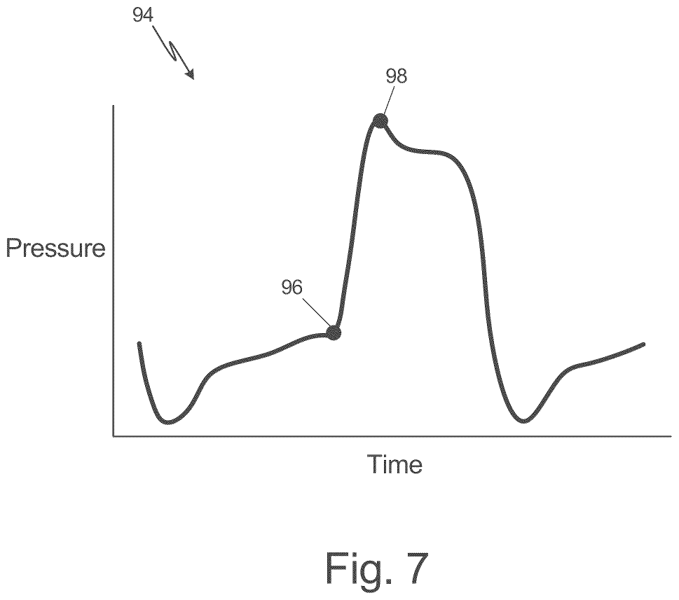

FIG. 7 is a graph illustrating an example trace of RV pressure waveform 94 corresponding to hemodynamic data sensed by one of hemodynamic sensors 16A and received by hemodynamic monitor 10. As further illustrated in FIG. 7, RV pressure waveform 94 (e.g., represented via digital hemodynamic data) can include various indicia corresponding to the presence and severity of RV systolic dysfunction in patient 70.

Prior to extracting indicia from RV pressure waveform 94, beat detector algorithms identify the start and end of individual heartbeats for RV pressure waveform 94. Right ventricular pressure beat detection algorithms identify the start of the heartbeat based on the maximum right ventricular pressure, the minimum right ventricular pressure, the maximum or minimum rate of change in right ventricular pressure, and/or the second derivative with respect to time in the right ventricular pressure. After heartbeat identification within RV pressure waveform 94, various indicia of RV systolic dysfunction can be extracted from the waveforms on an on-going, beat-to-beat basis.

FIG. 7 illustrates example indicia 96 and 98, corresponding respectively to end diastolic pressure (indicium 96) and peak systolic pressure (indicium 98) of the heartbeat of the patient. Additional indicia may be extracted from RV pressure waveform 94. System processor 74 executes RV systolic dysfunction software code 82 to determine the presence and/or severity of RV systolic dysfunction. Indicia 96 and 98 are extracted from RV pressure waveform 94 by waveform analysis module 84 of RV systolic dysfunction software code 82. Ratio generation module 86 of RV systolic dysfunction software code 82 calculates the ratio of peak systolic pressure (indicium 98) over end diastolic pressure (indicium 96). Additional indicia indicative of the start of a heartbeat can be extracted from RV pressure waveform 94 by RV systolic dysfunction software code 82.

RV systolic dysfunction is detected, and progression is assessed, based on the ratio of peak systolic pressure (indicium 98) over end diastolic pressure (indicium 96). RV systolic dysfunction results in a loss in systolic function, causing systolic pressure to decrease and/or end diastolic pressure to increase. As systolic pressure decreases and/or end diastolic pressure increases, the ratio of RV peak systolic pressure over RV end diastolic pressure decreases. As such, the lower the ratio of RV peak systolic pressure over RV end diastolic pressure, the more severe the RV systolic dysfunction. Because low ratios of RV peak systolic pressure over RV end diastolic pressure are not seen in other hemodynamic states, such as bleeding or fluid administration, using the ratio of RV peak systolic pressure over RV end diastolic pressure provides a more accurate assessment of RV systolic dysfunction.

Accordingly, hemodynamic monitor 10 provides information to medical personnel to detect RV systolic dysfunction, enabling timely and effective patient care. Moreover, hemodynamic monitor 10 enables assessment of the severity of RV systolic dysfunction. As the hemodynamic monitor 10 can continuously provide information regarding the severity of RV systolic dysfunction in patient 70, hemodynamic monitor 10 allows for RV systolic dysfunction to be constantly monitored. The usability of hemodynamic monitor 10 is increased as hemodynamic monitor 10 continuously monitors right ventricular systolic dysfunction in patient 70 to provide real-time updates of the detection of RV systolic dysfunction and/or an increase or decrease in the ratio of right ventricular peak systolic pressure over right ventricular end diastolic pressure signifying the severity of RV systolic dysfunction.

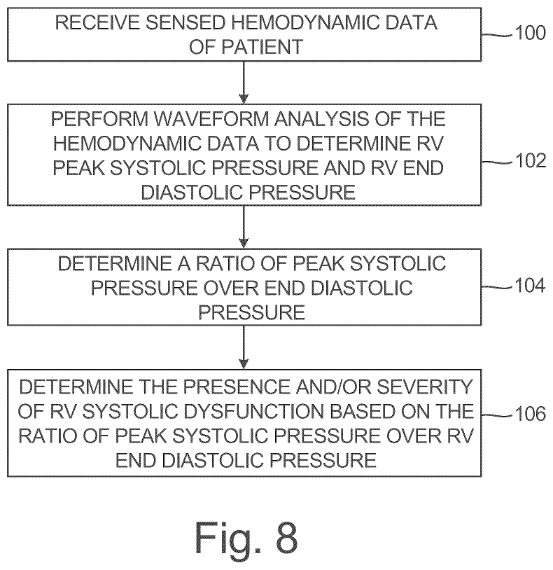

FIG. 8 is a flow diagram illustrating example operations to extract a set of features from an RV pressure waveform of a patient for detecting and assessing the progression of RV systolic dysfunction. For purposes of clarity and ease of discussion, the example operations are described below within the context of hemodynamic monitoring system 68 of FIG. 6.

Sensed hemodynamic data of patient 70 is received by hemodynamic monitor 10 (step 100). Sensed hemodynamic data is representative of RV pressure waveform 94 of patient 70. For instance, hemodynamic monitor 10 can receive an analog hemodynamic sensor signal representative of an RV pressure waveform of patient 70 from hemodynamic sensor 16A.

Hemodynamic monitor 10 performs waveform analysis of the hemodynamic data to determine RV systolic dysfunction profiling parameters, RV peak systolic pressure and RV end diastolic pressure (step 102). For example, hemodynamic monitor 10 can execute RV systolic dysfunction software code 82 to perform waveform analysis on an RV pressure waveform via waveform analysis module 84. Waveform analysis module 84 determines RV peak systolic pressure and RV end diastolic pressure of the RV pressure waveform. RV peak systolic pressure (indicium 98) and RV end diastolic pressure (indicium 96) are indicative of RV systolic dysfunction in patient 70.

Hemodynamic monitor 10 determines a ratio of peak systolic pressure over end diastolic pressure via RV systolic dysfunction software code 82 (step 104). For example, hemodynamic monitor 10 can execute ratio generation module 86 of RV systolic dysfunction software code 82. RV systolic dysfunction software code 82 determines a ratio of peak systolic pressure (indicium 98 in FIG. 7) over end diastolic pressure (indicium 96 in FIG. 7). The ratio of RV peak systolic pressure (indicium 98 in FIG. 7) over RV end diastolic pressure (indicium 96 in FIG. 7) are indicative of the presence and severity of RV systolic dysfunction.

The presence and/or severity of RV systolic dysfunction is determined based on the ratio of RV peak systolic pressure (indicium 98 in FIG. 7) over RV end diastolic pressure (indicium 96 in FIG. 7) (step 106). A ratio of RV peak systolic pressure (indicium 98 in FIG. 7) over RV end diastolic pressure (indicium 96 in FIG. 7) below 2.5 indicates RV systolic dysfunction. The lower the ratio of RV peak systolic pressure over RV end diastolic pressure, the more severe the RV systolic dysfunction. As the ratio of RV peak systolic pressure (indicium 98 in FIG. 7) over RV end diastolic pressure (indicum 96 in FIG. 7) decreases, RV systolic dysfunction increases. As the ratio of RV peak systolic pressure (indicium 98 in FIG. 7) over RV end diastolic pressure (indicium 96 in FIG. 7) increases, RV systolic dysfunction decreases. As such, the ratio of RV peak systolic pressure (indicium 98 in FIG. 7) over RV end diastolic pressure (indicium 96 in FIG. 7) allows medical personnel to detect and assess the progression of RV systolic dysfunction in a patient.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

A system for monitoring right ventricular systolic dysfunction in a patient includes a hemodynamic sensor that produces, on an ongoing basis, a hemodynamic sensor signal representative of a right ventricular pressure waveform of the patient; a display; one or more processors; and computer-readable memory encoded with instructions that, when executed by the one or more processors, cause the system to receive the hemodynamic sensor signal representative of the right ventricular pressure waveform of the patient; extract right ventricular peak systolic pressure and right ventricular end diastolic pressure from the right ventricular pressure waveform of the patient; determine a ratio of right ventricular peak systolic pressure over right ventricular end diastolic pressure; and output the ratio of right ventricular peak systolic pressure over right ventricular end diastolic pressure to the display for monitoring the presence of right ventricular systolic dysfunction based on the ratio of peak systolic pressure over end diastolic pressure.

The system of the preceding paragraph can optionally include, additionally and/or alternatively, any one or more of the following features, configurations and/or additional components:

Right ventricular systolic dysfunction is detected when the ratio of right ventricular peak systolic pressure over right ventricular end diastolic pressure is 2.5 or below.

Severe right ventricular systolic dysfunction is detected when the ratio of right ventricular peak systolic pressure over right ventricular end diastolic pressure is 2 or below.

The system continuously monitors right ventricular systolic dysfunction in the patient to provide real-time updates of the increase or decrease in the ratio of right ventricular peak systolic pressure over right ventricular end diastolic pressure.

The hemodynamic sensor is configured to be attached to a catheter.

The catheter includes a right ventricle port configured to be located within the right ventricle of a heart of the patient.

The one or more processors is configured to identify individual heartbeats for the right ventricular pressure waveform to allow for beat-to-beat monitoring.

A method for detecting right ventricular systolic dysfunction in a patient includes receiving, by a hemodynamic monitor, sensed hemodynamic data representative of a right ventricular pressure waveform of the patient; performing, by the hemodynamic monitor, waveform analysis of the hemodynamic data to determine right ventricular peak systolic pressure and right ventricular end diastolic pressure from the right ventricular pressure waveform of the patient; and determining, by the hemodynamic monitor, a ratio of right ventricular peak systolic pressure over right ventricular end diastolic pressure to detect right ventricular systolic dysfunction.

The method of the preceding paragraph can optionally include, additionally and/or alternatively, any one or more of the following features, configurations and/or additional components:

Right ventricular systolic dysfunction is detected when the ratio of right ventricular peak systolic pressure over right ventricular end diastolic pressure is 2.5 or below.

Severe right ventricular systolic dysfunction is detected when the ratio of right ventricular peak systolic pressure over right ventricular end diastolic pressure is 2 or below.

Receiving sensed hemodynamic data includes receiving sensed hemodynamic data representative of a right ventricular pressure waveform of the patient on an ongoing basis to continuously monitor right ventricular systolic dysfunction in the patient and detect an increase or a decrease in the ratio of right ventricular peak systolic pressure over right ventricular end diastolic pressure.

Connecting the hemodynamic sensor to a catheter.

The catheter includes a right ventricle port configured to be located within the right ventricle of a heart of the patient.

Performing waveform analysis of the hemodynamic data includes identifying individual heartbeats for the right ventricular pressure waveform to allow for beat-to-beat monitoring of right ventricular systolic dysfunction.

A system for detecting right ventricular systolic dysfunction in a patient includes a hemodynamic sensor that produces a hemodynamic sensor signal representative of a right ventricular pressure waveform of the patient; a display; one or more processors; and computer-readable memory encoded with instructions that, when executed by the one or more processors, cause the system to: receive the hemodynamic sensor signal representative of the right ventricular pressure waveform of the patient; extract right ventricular peak systolic pressure and right ventricular end diastolic pressure from the right ventricular pressure waveform of the patient; determine a ratio of right ventricular peak systolic pressure over right ventricular end diastolic pressure; and output the ratio of right ventricular peak systolic pressure over right ventricular end diastolic pressure to the display for detecting right ventricular systolic dysfunction based on the ratio of peak systolic pressure over end diastolic pressure.

The system of the preceding paragraph can optionally include, additionally and/or alternatively, any one or more of the following features, configurations and/or additional components:

Right ventricular systolic dysfunction is detected when the ratio of right ventricular peak systolic pressure over right ventricular end diastolic pressure is 2.5 or below.

Severe right ventricular systolic dysfunction is detected when the ratio of right ventricular peak systolic pressure over right ventricular end diastolic pressure is 2 or below.

The hemodynamic sensor is configured to be attached to a catheter.

The catheter includes a right ventricle port configured to be located within the right ventricle of a heart of the patient.

The one or more processors is configured to identify individual heartbeats for the right ventricular pressure waveform.

The above method(s) can be performed on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for monitoring right ventricular systolic dysfunction in a patient, the system comprising:
a hemodynamic sensor attached to a right-heart catheter with a includes a right ventricle port located approximately 30 cm from a tip of the right-heart catheter and configured to be located within a right ventricle of a heart of the patient, wherein the hemodynamic sensor produces, on an ongoing basis, a hemodynamic sensor signal representative of a right ventricular pressure waveform of the patient;
a display;
a sensory alarm;
one or more processors; and
computer-readable memory encoded with instructions that, when executed by the one or more processors, cause the system to:
receive the hemodynamic sensor signal representative of the right ventricular pressure waveform of the patient;
extract right ventricular peak systolic pressure and right ventricular end diastolic pressure from the right ventricular pressure waveform of the patient;
determine a ratio of right ventricular peak systolic pressure over right ventricular end diastolic pressure; and
output the ratio of right ventricular peak systolic pressure over right ventricular end diastolic pressure to the display for monitoring a presence of right ventricular systolic dysfunction based on the ratio of peak systolic pressure over end diastolic pressure; and
activate the sensory alarm if the ratio is below a threshold.

2. The system of claim 1, wherein the presence of right ventricular systolic dysfunction is detected when the ratio of right ventricular peak systolic pressure over right ventricular end diastolic pressure is 2.5 or below.

3. The system of claim 2, wherein severe right ventricular systolic dysfunction is detected when the ratio of right ventricular peak systolic pressure over right ventricular end diastolic pressure is 2 or below.

4. The system of claim 1, wherein the system continuously monitors right ventricular systolic dysfunction in the patient to provide real-time updates of the increase or decrease in the ratio of right ventricular peak systolic pressure over right ventricular end diastolic pressure.

5. The system of claim 1, wherein the hemodynamic sensor is a DPT configured to be attached to a lumen of the right-heart catheter leading to the right ventricular port.

6. The system of claim 5, wherein the right-heart catheter does not include a thermal filament or corresponding thermal filament connector.

7. The system of claim 1, wherein the one or more processors is configured to identify individual heartbeats for the right ventricular pressure waveform to allow for beat-to-beat monitoring.

8. A method for detecting right ventricular systolic dysfunction in a patient, the method comprising:
receiving, by a hemodynamic monitor, sensed hemodynamic data representative of a right ventricular pressure waveform of the patient;
performing, by the hemodynamic monitor, waveform analysis of the hemodynamic data to determine right ventricular peak systolic pressure and right ventricular end diastolic pressure from the right ventricular pressure waveform of the patient; and
determining, by the hemodynamic monitor, a ratio of right ventricular peak systolic pressure over right ventricular end diastolic pressure to detect right ventricular systolic dysfunction.

9. The method of claim 8, wherein the presence of right ventricular systolic dysfunction is detected when the ratio of right ventricular peak systolic pressure over right ventricular end diastolic pressure is 2.5 or below.

10. The method of claim 9, wherein severe right ventricular systolic dysfunction is detected when the ratio of right ventricular peak systolic pressure over right ventricular end diastolic pressure is 2 or below.

11. The method of claim 8, wherein receiving sensed hemodynamic data includes receiving sensed hemodynamic data representative of the right ventricular pressure waveform of the patient on an ongoing basis to continuously monitor right ventricular systolic dysfunction in the patient and detect an increase or a decrease in the ratio of right ventricular peak systolic pressure over right ventricular end diastolic pressure.

12. The method of claim 8, further including connecting a hemodynamic sensor to a catheter.

13. The method of claim 12, wherein the catheter includes a right ventricle port configured to be located within a right ventricle of a heart of the patient.

14. The method of claim 8, wherein performing waveform analysis of the hemodynamic data includes identifying individual heartbeats for the right ventricular pressure waveform to allow for beat-to-beat monitoring of right ventricular systolic dysfunction.

15. A system for detecting right ventricular systolic dysfunction in a patient, the system comprising:
a hemodynamic sensor that produces a hemodynamic sensor signal representative of a right ventricular pressure waveform of the patient;
a display;
one or more processors; and
computer-readable memory encoded with instructions that, when executed by the one or more processors, cause the system to:
receive the hemodynamic sensor signal representative of the right ventricular pressure waveform of the patient;
extract right ventricular peak systolic pressure and right ventricular end diastolic pressure from the right ventricular pressure waveform of the patient;
determine a ratio of right ventricular peak systolic pressure over right ventricular end diastolic pressure; and
output the ratio of right ventricular peak systolic pressure over right ventricular end diastolic pressure to the display for detecting right ventricular systolic dysfunction based on the ratio of peak systolic pressure over end diastolic pressure.

16. The system of claim 15, wherein right ventricular systolic dysfunction is detected when the ratio of right ventricular peak systolic pressure over right ventricular end diastolic pressure is 2.5 or below.

17. The system of claim 16, wherein severe right ventricular systolic dysfunction is detected when the ratio of right ventricular peak systolic pressure over right ventricular end diastolic pressure is 2 or below.

18. The system of claim 15, wherein the hemodynamic sensor is configured to be attached to a catheter.

19. The system of claim 18, wherein the catheter includes a right ventricle port configured to be located within a right ventricle of a heart of the patient.

20. The system of claim 15, wherein the one or more processors is configured to identify individual heartbeats for the right ventricular pressure waveform.
